(19) United States
(12) Patent Application Publication
(10) Pub. No.: US 2024/0408028 A1
(43) Pub. Date: Dec. 12, 2024

(54) COATED ACTIVE INGREDIENT FORMULATION FOR CONTROLLED IN-VIVO UPTAKE OF CO2

(71) Applicants: O11-biomedical GmbH, Aachen (DE); RHEINISCH WESTFÄLISCHE TECHNISCHE HOCHSCHULE (RWTH) AACHEN, Aachen (DE)

(72) Inventors: Stefan JOCKENHÖVEL, Aachen (DE); Richard RAMAKERS, Aachen (DE); Christian CORNELISSEN, Aachen (DE)

(21) Appl. No.: 18/702,835

(22) PCT Filed: Oct. 25, 2022
(86) PCT No.: PCT/EP2022/079779

§ 371 Date: Apr. 19, 2024

(30) Foreign Application Priority Data
Oct. 27, 2021 (DE) 10 2021 127 957.7
Sep. 21, 2022 (DE) 10 2022 124 227.7

(51) Int. Cl. A61K 9/50; A61K 33/08; A61P 11/00

(52) U.S. Cl. CPC A61K 9/5031; A61K 9/5073; A61K 9/5089; A61K 33/08; A61P 11/00

(57) ABSTRACT

The present invention relates to a coated active ingredient formulation for the controlled in-vivo uptake of CO2, and more specifically in the form of microspherules. The invention further relates to a liquid medical composition containing the active ingredient formulation, and to its use for treating selected diseases, in particular respiratory diseases. The invention finally relates to a method for producing microspherules with a controlled CO2 uptake.

The present invention relates to a coated active ingredient formulation for the controlled in vivo uptake of CO₂, and in this case in particular in the form of microspherules. The invention furthermore relates to a liquid medical composition which contains said active ingredient formulation, and to the use thereof for treating selected diseases, in particular in the respiratory field. Finally, the invention also relates to a method for producing microspherules that accept CO₂ in a controlled manner.

### BACKGROUND OF THE INVENTION

Numerous respiratory diseases exist, which are associated with an increased content of CO₂ in the blood, which is also referred to as hypercapnia. The cause of hypercapnia is usually a disruption of the pulmonary ventilation (formerly referred to as global respiratory failure), which occurs on account of alveolar hypoventilation, pulmonary diffusion disorder. Hypercapnic respiratory failure can occur for example in the case of worsening of chronic obstructive pulmonary disease (COPD). However, hypercapnia also results from metabolic alkalosis or from breathing in air that contains a lot of carbon dioxide (from 8 to 10 volume percent carbon dioxide poisoning occurs). Medical practitioners also refer to hypercapnic respiratory failure, i.e. carbon dioxide poisoning, together with simultaneous lack of oxygen in the blood. Typical signs of hypercapnia are breathlessness, restlessness but also tiredness, headaches, confusion, bluish skin, and a raised pulse.

For treating hypercapnic respiratory failure, in order to assist the breath work the patient can be mechanically ventilated, for example by means of pressure-assisted, non-invasive ventilation. Treatment of this kind is device- and cost-intensive and is associated with numerous risks, such as damage to the lungs due to pressure, pneumonia, increase in pressure in the thoracic cavity, or worsening of cardiac insufficiency.

However, hypercapnia can also occur in the course of intensive medical treatment of children and adults, when increased CO₂ particle pressures are accepted, in favour of more gentle ventilation (also referred to as “permissive hypercapnia”).

There is therefore a need for alternative methods for treating hypercapnia. For example, there are experimental approaches aiming to reduce the CO₂ content in the blood by oral administration of CO₂ absorbers, exploiting the large inner surface area of the human digestive system.

In this respect, WO 2020/035163 A1 discloses a composition consisting of a CO₂ absorbent or adsorbent having a polymer coating made of a silicone rubber produced from liquid silicone rubber, or having a polymer coating made from a cellulose derivate. For this purpose, it teaches rubber-coating by simple dip coating using liquid silicone rubber, and subsequent compression to form tablets. This has the disadvantage that organic solvents that are harmful to health have to be used, and particles having a non-uniform coating result, such that the absorption kinetics cannot be predicted. Furthermore, the method is not suitable for large-scale production. Furthermore, the silicone-rubber particles have a tacky surface, which leads to agglomeration and makes production of a doseable formulation more difficult.

Alternatively, WO'163 teaches encapsulation of a powder CO₂ absorber in a capsule made of regenerated cellulose. A capsule of this kind is associated with several disadvantages. Regenerated cellulose has only low CO₂ permeability and, on account of its hygroscopic behaviour, leads to swelling and bad pouring behaviour even in the liquid formulation, which is associated with a reduced shelf life. Corresponding capsules do not have the sufficient strength that is required for the inclusion of highly basic absorber substances. In this case, slight damage to the capsule shell leads immediately to “dose dumping”, i.e. the sudden and wholesale release of CO₂ absorbers into the organism. Furthermore, the production method is difficult to scale to the required amounts.

Further prior art can be found in WO 2005/097075 A2, US 2015/245999 A1 or US 2017/319617 A1.

The problem addressed by the present invention is that of improving the prior art or providing an alternative.

### SUMMARY OF THE INVENTION

According to a first aspect of the present invention, the problem addressed is solved by a coated active ingredient formulation for the controlled in vivo uptake of CO₂, wherein the coated active ingredient formulation comprises the following:
(i) a coating that consists of a wall material, wherein the coating is permeable for CO₂ and optionally also for water vapour;
(ii) and a core region which comprises at least one CO₂-accepting active ingredient.

The further independent and dependent claims relate to further embodiments.

With regard to terminology, the following is to be explained in this respect:

The invention firstly relates to a coated active ingredient formulation. A CO₂-accepting active ingredient is provided as the active ingredient in the formulation. The mechanism of action correspondingly consists in the in vivo uptake of carbon dioxide. In the context of the present invention, the in vivo uptake of CO₂ means an uptake of CO₂ in living organism as a whole, wherein extrapulmonary reduction of carbon dioxide takes place. Correspondingly, the active ingredient formulation must also be suitable for in vivo application.

Furthermore, the active ingredient formulation according to the invention achieves a controlled uptake of CO₂. This is achieved on the one hand by a separation into a CO₂-accepting core region and a coating that surrounds said core, and on the other hand by the design of the coating as being permeable for CO₂.

In the case of a basic CO₂ absorber, due to the water vapour permeability of the coating the basic CO₂ absorber is activated only in conjunction with the resulting wetting. In this case, the carbon dioxide is converted, by the small amount of water, into carbonic acid, which then reacts with the CO₂ absorber to form a carbonate compound and water.

The invention has a plurality of advantages compared with the prior art.

The production method in the context of the invention makes it possible to produce formulations which have an extremely homogeneous particle size distribution. This ensures a purposeful controllability of the CO₂-uptake by the active ingredient.

Furthermore, the coated active ingredient formulation of the invention, in particular as a microspherule, is available for large-scale production. The disparate separation into the core region and coating region makes it possible for the absorption capacity and/or the absorption kinetics to be purposefully controlled.

As a result, active ingredient formulations can thus be produced which are readily distributable and/or are storage-stable. Their properties sort in the end formulations can thus be achieved without problem.

On account of its enteral administration, the present coated active ingredient formulation can be used widely for treatment of respiratory diseases, without impairing or putting strain on the respiratory tract.

An innovative treatment option that is low in side-effects results, which can even be used for newborns, children, and high-risk patients.

### The Invention in Detail

It can advantageously be provided, in the case of the coated active ingredient formulation according to the invention, that the coating is impermeable to water. This is advantageous both for the preparation of the formulation (e.g. as an aqueous suspension) and also for the in vivo application. Although, in one embodiment, the coating is permeable for water vapour and thus allows controlled wetting of the active ingredient, penetration of water into the core region is thus prevented. By reaction with water, the core could swell and damage the coating, or at least lose its CO₂-accepting properties due to the reaction with water.

In the context of the present application, the term “impermeability to water” is synonymous with the term “watertightness”. What is referred to as the watertightness of the coating is measured on the basis of head of water, under the pressure of which the material begins to let water through. From a value of 1300 mm upwards a membrane is impermeable to water according to EN 343.

The coated active ingredient formulation can be provided in one of the following four embodiments:

Embodiment 1: Liquid core comprising the CO₂-accepting active ingredient having a membrane envelope for forming a liposome or niosome.

In one embodiment, the coated active ingredient formulation can be present as a liposome. The advantage of liposomes is in the high level of gas-permeability of the membrane envelope, such that high CO₂-uptake is possible on the coating side. Niosomes are microscopically small balls that are similar to the liposomes and in which a double membrane surrounds an interior. Active ingredients can be incorporated into this interior. Niosomes are constructed analogously to the liposomes. However, liposomes contain double membranes formed of phospholipids, whereas the building blocks of the niosome double membrane are derived from sugars or amino acids.

Embodiment 2: Solid core comprising a CO₂-accepting active ingredient having a single-layer coating.

In this embodiment, the solid core is coated with a single-layer coating. Accordingly, this is a formulation that is of simple construction and is cost-effective to produce.

Embodiment 3: Solid core comprising a CO₂-accepting active ingredient having a multi-layer coating.

In this embodiment, the solid core is coated with a multi-layer coating. This is a formulation that is of more complex construction and in which the properties of the coating with respect to permeability, stability or biocompatibility can be purposely set or distributed. For example, an inner coating layer can provide the formulation with the necessary strength, and the prescribed permeability can be achieved by an outer coating layer.

Embodiment 4: Microcapsule.

In this embodiment, the coated active ingredient formulation can also be present as a microcapsule.

Microcapsules are pourable powder having particle diameters in the range of approximately 1 μm-1000 μm. They are produced using different coating processes of finely distributed solid or liquid substances. Typically polymers are used as the coating or wall material. In principle, microcapsules thus consist of two disparate regions-the core region and the coating region.

In order for the microcapsules to be pourable, no adhesive forces may occur between the particles, which forces entail adhesion of the particles or even agglomeration (“clumping”). The pourability can be determined on the basis of the DIN Standard EN ISO 6186, as is also described in the European Pharmacopoeia, Chapter 2.9.1 “Flowability”. This defines apparatus used for monitoring and testing the pourability of solids in powder or granulate form (for example plastics granulate). In this case, the material is dispensed using a standardised funnel, and the elapsed time is determined using a stopwatch. Information relating to the processability and the uniform process control during production can be derived. Good pourability is important for trouble-free automatic processing. The pourability of materials is substantially influenced by the surface condition of the grains.

Production methods that are suitable for microencapsulation are phase-separation methods (simple and complex coacervation), interfacial polymerisation methods (polycondensation or polyaddition from dispersions), and mechanical/physical methods (fluidised bed methods, spray drying).

The fundamental disadvantage in conventional microencapsulation is the relatively high production outlay.

It can advantageously be provided, in the case of the invention, that, in the coated active ingredient formulation, the microcapsule is a microspherule, in which the CO₂-accepting active ingredient is incorporated in solid dispersed form or in dissolved form in a polymer carrier matrix.

Microspherules are systems related to microcapsules, in which there is no exact separation into core and coating region. However, with respect to their size of approximately 1 μm-1000 μm microspherules correspond to the microcapsules. In microspherules, the embedded active ingredient is present incorporated in the carrier matrix in solid, i.e. dispersed, or dissolved form. Microspherules are thus a special type of microcapsules. The carrier matrix typically consists of a polymer.

The embedding of the CO₂-accepting active ingredient in microspherules has the advantage that the carrier matrix provides a continuous spectrum with respect to the “wall thickness” (in this case defined as the shortest distance from the active ingredient to the outside of the microspherule), such that the penetrating CO₂ gas accordingly strikes the active ingredient in a temporally staggered manner, starting at the active ingredient inclusions closest to the surface. This results in advantageous CO₂-uptake kinematics, which, in the case of early onset, leads to a long-lasting continuous CO₂ uptake.

It can be provided, in the case of the invention, that, in the case of the coated active ingredient formulation, the CO₂-accepting active ingredient is selected from the group consisting of CO₂-absorbing material, CO₂-adsorbing material, CO₂-dissolving material, and CO₂-converting enzyme, and is preferably a CO₂-absorbing material.

The term “absorption” describes the process of the uptake or the “dissolving” of the carbon dioxide in another phase. This is not an accumulation on the surface (adsorption), but rather an uptake into the free volume of the absorbing phase. In the case of absorption, a distinction is made between physical and chemical absorption. In the case of physical absorption, the carbon dioxide is dissolved, as a gas, in a solvent. Mixing takes place, without chemical reaction.

In the case of chemical absorption, the carbon dioxide undergoes a chemical reaction with the “solvent” or absorber, such that a product substance is formed.

In the present case, “adsorption” refers to the concentration of the carbon dioxide at the surface of a solid.

It can advantageously be provided, in the case of the invention, that, in the coated active ingredient formulation, the coating has a CO₂ permeability of 0.004 to 2,500×10⁻¹³ cm³ cm cm⁻² Pa⁻¹ s⁻¹, preferably of 5 to 500×10⁻¹³ cm³ cm cm⁻² Pa⁻¹ s⁻¹, and particularly preferably of 10 to 300×10⁻¹³ cm³ cm cm⁻² Pa⁻¹ s⁻¹. The coating can for example have a CO₂ permeability of 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250 or 275×10⁻¹³ cm³ cm cm⁻² Pa⁻¹ s⁻¹. It has been found that a coating having a CO₂ permeability of this kind achieves adequate profiles with respect to the absorption kinetics.

Suitably, a material, which is preferably a polymer, having high permeability for CO₂ is used as the wall material. This allows efficient CO₂ uptake in the context of in vivo application. By way of example, in this respect the following polymers are mentioned: Polyethylene such as high-density polyethylene (HD-PE) or low-density polyethylene (LD-PE), polylactide (PLA), polybutylene adipate terephthalate (PBAT, EcoFlex), and polyhydroxyalkanoates such as poly[(R)-3-hydroxybutyrate] (PHB).

The CO₂-permeable coating defined above is preferably the carrier matrix of a microspherule.

It can advantageously be provided, in the case of the invention, that, in the coated active ingredient formulation, the coating has a water vapour permeability of 0.009 to 32,000×10⁻¹³ cm³ cm cm⁻² Pa⁻¹ s⁻¹, and preferably of 100 to 1500×10⁻¹³ cm³ cm cm⁻² Pa⁻¹ s⁻¹. The coating can for example have a water vapour-permeability of 150, 200, 250, 300, 350, 400, 450, 500, 550, 600, 650, 700, 750, 800, 850, 900, 950, 1000, 1050, 1100, 1150, 1200, 1250, 1300, 1350, 1400 or 1450×10⁻¹³ cm³ cm cm⁻² Pa⁻¹ s⁻¹. It has been found that a coating having a water vapour permeability of this kind allows wetting of the absorber material up to approximately 10 wt. % moisture, which is essential for the efficient absorption of CO₂, precisely in the case of basic absorbers such as calcium hydroxide, since only after reaction of the carbon dioxide to form carbonic acid can this carbonic acid react with the calcium hydroxide to form calcium carbonate and water.

Suitably, a material, which is preferably a polymer, having sufficiently high permeability for water vapour is used as the wall material. This allows for CO₂ absorption, taking place via carbonic acid occurring as an intermediate, in the context of in vivo application. By way of example, in this respect the following polymers are mentioned: Polyethylene such as high-density polyethylene (HD-PE) or low-density polyethylene (LD-PE), polylactide (PLA), polybutylene adipate terephthalate (PBAT, EcoFlex), and polyhydroxyalkanoates such as poly[(R)-3-hydroxybutyrate] (PHB).

The water vapour-permeable coating defined above is preferably the carrier matrix of a microspherule.

In a further possibility, it can be provided that, in the case of the coated active ingredient formulation, the coating has a mass fraction of 10 to 95 wt. %, preferably of 40 to 60 wt. %, and particularly preferably of 55 to 65 wt. %, based on the weight of the coated active ingredient formulation. The mass fraction of the coating can for example be 42, 44, 46, 48, 50, 52, 54, 56, 58, 60, 62 or 64 wt. %. In this case, the coating is preferably the carrier matrix of a microspherule.

It is furthermore conceivable that, in the case of the coated active ingredient formulation, the coating has an average layer thickness of 10 to 500 μm, preferably of 50 to 250 μm, and particularly preferably of 100 to 200 μm. As has been found, layer thicknesses of this kind on the one hand still allow sufficient CO₂-uptake, but on the other hand are stable enough to prevent undesired release of the core material into the organism.

In the case of the coated active ingredient formulation, the wall material forming the coating is preferably a polymer, and particularly preferably selected from the group consisting of poly(organo)siloxane; polyolefin; polyester; polybutylene succinate; polybutylene adipate terephthalate (PBAT); polyethylene such as high-density polyethylene (HDPE) or low-density polyethylene (LDPE); polylactide (PLA), poly(glycolic acid) (PGA), and poly-lactic-co-glycolic-acid (PLGA); lipids such as fatty acids or phospholipids. In this case, the wall material is particularly preferably a polyester. The use of a polyester has the advantage that it exhibits very good resistance to bases and acids, and can be processed in a particularly wide process window.

Suitably, a pharmaceutically harmless material, which is preferably a polymer, is used as the wall material. This allows for in vivo application that has few side-effects or no side-effects. By way of example, in this respect the following polymers are mentioned: Polyethylene such as high-density polyethylene (HD-PE) or low-density polyethylene (LD-PE), polylactide (PLA), polybutylene adipate terephthalate (PBAT, EcoFlex), and polyhydroxyalkanoates such as poly[(R)-3-hydroxybutyrate] (PHB).

Suitably, a material (preferably in a polymer) is used as the wall material which, on account of its material purity, has a content of what are known as “leachables” or “extractables” that is below the admissible limit values. This allows for in vivo application that has few side-effects or no side-effects.

In the case of the coated active ingredient formulation, the coating is suitably inert with respect to the CO₂-accepting active ingredient. That is to say that in the case of a basic CO₂-accepting active ingredient, such as calcium hydroxide, the coating is base-stable.

In the context of the present invention the term “stable”, when used for characterising the coating in a base, means that the coating remains largely completely intact, after a solution of saturated calcium hydroxide solution acts either over one day at 90° C. or 30 days at 40° C., the coating preferably remains substantially completely intact under these conditions, and the coating particularly preferably remains virtually completely intact under these conditions. In the context of acid treatment, the terms “largely, substantially completely and virtually completely” mean that the coating matrix retains at least 90%, at least 95%, and at least 99%, respectively, of its chemical bonds in the polymer scaffold after action of these conditions.

That is to say, furthermore, that in the case of an acidic CO₂-accepting active ingredient the coating is acid-stable. The acid-stability is also to be demanded in the case of oral administration or passage through the stomach.

In the context of the present invention the term “stable”, when used for characterising the coating in an acid, means that the coating remains largely completely intact, after a solution of 20% sulfuric acid acts either over one day at 90° C. or 30 days at 40° C., the coating preferably remains substantially completely intact under these conditions, and the coating particularly preferably remains virtually completely intact under these conditions. In the context of acid treatment, the terms “largely, substantially completely and virtually completely” mean that the coating matrix retains at least 90%, at least 95%, and at least 99%, respectively, of its chemical bonds in the polymer scaffold after action of these conditions.

It is furthermore advantageous if, in the case of the coated active ingredient formulation, the coating forms a semipermeable barrier, insofar as nonpolar molecules such as CO₂ gas molecules can pass the coating in order to thereby reach the CO₂-accepting active ingredient, in the core region. Said coating, as a semipermeable barrier, is, however, impermeable for charged molecules such as salt cations or anions, and correspondingly also protons and hydroxide anions. Thus, the coating represents a selectively permeable coating; this selective permeability makes it possible to prevent, for example in the case of a highly basic CO₂-absorbing material such as calcium hydroxide, a discharge of hydroxide ions into the organism from occurring.

Wall materials which enable the construction of such a selectively permeable coating, described above, are for example polyethylene such as high-density polyethylene (HD-PE) or low-density polyethylene (LD-PE), polylactide (PLA), polybutylene adipate terephthalate (PBAT, EcoFlex), and polyhydroxyalkanoates such as poly[(R)-3-hydroxybutyrate] (PHB).

It can advantageously be provided, in the invention, that the CO₂-accepting active ingredient is one of the following materials:
(a) CO₂-absorbing material selected from the group consisting of inorganic hydroxide compounds, lithium peroxide, and mixtures thereof, wherein lithium hydroxide, sodium hydroxide, potassium hydroxide, calcium hydroxide, and magnesium hydroxide, and mixtures thereof, are preferred; or
(b) CO₂-adsorbing material selected from the group consisting of active carbon, molecular sieve such as zeolite, silica, metalorganic frameworks (MOFs); or
(c) CO₂-dissolving material selected from the group consisting of highly-eutectic solvent and ionic fluid; or
(d) CO₂-converting enzyme such as carbonic anhydrase (EC 4.2.1.1).

It can advantageously be provided, in the invention, that the CO₂-absorbing material comprises calcium hydroxide.

According to a further embodiment, the CO₂-absorbing material substantially consists of calcium hydroxide. The calcium hydroxide reacts with the carbon dioxide to form pharmaceutically harmless calcium carbonate. In this case, “substantially” means that the CO₂-absorbing material contains at most 5 wt. %, preferably at most 4 wt. %, preferably at most 3 wt. %, more preferably at most 1 wt. %, of other components.

In an advantageous embodiment it is provided that the calcium hydroxide-containing, CO₂-absorbing material is substantially free of sodium and potassium hydroxide. In this case, “substantially” means that the CO₂-absorbing material contains at most 4 wt. %, preferably at most 2 wt. %, preferably at most 1 wt. %, more preferably at most 0.5 wt. %, of sodium and potassium hydroxide, and very particularly preferably no sodium and potassium hydroxide.

In one embodiment, the calcium hydroxide-containing compositions described above contain, as an additional optional component, an effective amount, capable of absorption of carbon dioxide, of a hygroscopic or deliquescent humectant.

According to a further embodiment, the CO₂-absorbing material substantially consists of magnesium hydroxide. Magnesium hydroxide has the advantage that, on account of the low basicity, it is associated with improved tolerability in the case of gastrointestinal application. Furthermore, on account of its hygroscopic nature it facilitates initial wetting of the absorber material, which, by means of the solution of the CO₂ as carbonic acid, promotes the absorption, forming the carbonate. Finally, it is a cost-effective substance, and it has been found that it can be processed well, specifically in the production of microspherules. The magnesium hydroxide reacts with the carbon dioxide to form pharmaceutically harmless magnesium carbonate. In this case, “substantially” means that the CO₂-absorbing material contains at most 5 wt. %, preferably at most 4 wt. %, preferably at most 3 wt. %, more preferably at most 1 wt. %, of other components.

In an advantageous embodiment it is provided that the magnesium hydroxide-containing, CO₂-absorbing material is substantially free of sodium and potassium hydroxide. In this case, “substantially” means that the CO₂-absorbing material contains at most 4 wt. %, preferably at most 2 wt. %, preferably at most 1 wt. %, more preferably at most 0.5 wt. %, of sodium and potassium hydroxide, and very particularly preferably no sodium and potassium hydroxide.

In one embodiment, the magnesium hydroxide-containing compositions described above contain, as an additional optional component, an effective amount, capable of absorption of carbon dioxide, of a hygroscopic or deliquescent humectant.

In the case of the coated active ingredient formulation, it can advantageously be provided that CO₂-adsorbing material is selected from the group consisting of active carbon, a molecular sieve such as zeolite, silica, and metalorganic frameworks (MOFs).

Active carbon is a fine-grained carbon having a large inner surface area which can be between 300 and 2000 m²/g carbon. In the present case, the term “molecular sieve” is the functional designation for natural and synthetic zeolites or other substances which have a high adsorption capacity for the carbon dioxide gas. In addition to zeolites, there are for example also carbon molecular sieves or molecular sieving carbon. The molecular sieves have a large inner surface area (600-700 m²/g) and have a uniform pore diameter in the order of magnitude of the diameters of molecules. Metalorganic frameworks (MOFs) are defined, according to the present application, as microporous materials which are constructed from what are known as inorganic building units (IBUs) and organic molecules as linkers between the inorganic building units. MOFs are coordination networks having an open framework which contains pores. The pores of the three-dimensional structures are usually filled with guest molecules (e.g. solvent or unreacted linker) after the synthesis. By removing the guest molecules (e.g. by baking out, in a vacuum, or by a combination of the two) the pores can be made accessible again for uptake of gases such as CO₂.

In the context of the present application, deep eutectic solvents (DES) refers to multi-component, eutectic molten salts, the melting point of which, like that of ionic liquids, is close to or below room temperature. Examples for eutectic solvents are based on a mixture of quaternary ammonium compounds with hydrogen bridge bond donors (e.g. amines) and carboxylic acid.

According to a further advantage, it can be provided that, in the case of the active ingredient formulation according to the invention, the CO₂-absorbing material has a CO₂ acceptance capacity of more than 10 mg CO₂/g active ingredient formulation, preferably of more than 40 mg CO₂/g active ingredient formulation, particularly preferably of more than 50 mg CO₂/g active ingredient formulation, and in particular of more than 75 mg CO₂/g active ingredient formulation. The higher the CO₂ acceptance capacity, the smaller amounts of applied active ingredient formulation are required in order to achieve a therapeutically relevant effect.

In a second aspect, the invention relates to a liquid medical composition which comprises, as a dispersion, the coated active ingredient formulation according to the invention and a pharmaceutically harmless aqueous carrier fluid.

A “medical composition” within the meaning of the invention is a composition which can be used for prevention, diagnosis, treatment or relief of diseases. It thus includes both pharmaceutical compositions for preparing a medication, and also compositions for preparing a medicine product. It also further comprises compositions for non-medical use, insofar as they are suitable for application to mammals, and in particular humans.

A dispersion is defined, in the context of the application, as a heterogeneous mixture of two substances which do not or barely dissolve in one another or bond chemically with one another. In this case, the active ingredient formulation is present as a disperse phase distributed in the carrier fluid as a dispersion medium.

In the case of the use, according to the invention, of the coated active ingredient formulation or the liquid medical composition for treatment of diseases of the respiratory system, such as acute or chronic respiratory diseases or lung diseases; of cardiovascular diseases, or of infectious diseases, or respiratory disorders following serious disease processes, these diseases can occur both as main diseases and as concomitant diseases.

Furthermore, the coated active ingredient formulation or the liquid medical composition produced therefrom can also be used for weaning from medical ventilation, i.e. in the phase of waning of a ventilated patient from the ventilator.

In a further aspect, the invention relates to the liquid medical composition according to the invention in its use in intraperitoneal or enteral application, for example orally, rectally or via a tube.

The advantageous properties of the present coated active ingredient formulation with respect to controlled and efficient CO₂-uptake make it possible to use this formulation in a broad spectrum of application forms.

An enteral application is preferred here, i.e. an application which achieves the effect via the digestive tract. In this case, the enteral application can take place orally but also perorally, for example distally of the oral cavity via a tube, via a stoma, or rectally. Since, depending on the extent of the hypercapnia, sometimes large amounts of the formulation according to the invention have to be applied, an enteral application is advantageous, which also allows for simple exchange for a new active ingredient formulation after completed CO₂ uptake, by means of rinsing processes.

Furthermore, the active ingredient formulation can also be applied as intraperitoneal application.

In a preferred embodiment, the oral application is taken out of the application forms, and so the active ingredient formulation is precisely not taken in via the mouth, but rather supplied to the organism in other ways (such as by rectal application).

The invention furthermore relates to the coated active ingredient formulation or the liquid medical composition produced therewith for use in the prophylaxis or treatment of diseases of the respiratory system, such as acute or chronic respiratory diseases or lung diseases; of cardiovascular diseases, metabolic derailments which are associated with acidosis (e.g. ketoacidosis), or of infectious diseases, or respiratory disorders following serious disease processes.

In this case, the disease of the respiratory system is preferably selected from the list consisting of:
(a) chronic obstructive pulmonary disease;
(b) asthma;
(c) cystic fibrosis;
(d) acute respiratory distress syndrome;
(e) hypercapnia;
(f) pneumonia;
(g) lung cancer;
(h) pulmonary fibrosis;
(i) respiratory disorders according to medical provisions pursuant to ICD-10 J95;
(j) respiratory failure pursuant to ICD-10 J96;
(i) other respiratory disorders pursuant to ICD-10 J97;
(l) respiratory disorders in diseases classified elsewhere pursuant to ICD-10, J99;
(m) immature lungs.

The invention furthermore relates to the coated active ingredient formulation or the liquid medical composition produced therewith for use in the induction of hypocapnia in a patient. This includes possible uses that require extrapulmonary reduction of the CO₂ content in the body. Thus, the coated active ingredient formulation makes it possible to actively or passively reduce the intracranial pressure without hyperventilation, because a reduced CO₂ particle pressure has a vasoactive effect.

In this case, it is preferred for the coated active ingredient formulation to be present in the form of microspherules.

In an additional aspect, the invention relates to the use of the coated active ingredient formulation according to the invention or the liquid medical composition according to the invention for increasing performance in physical exertion or reducing the recovery time after physical exertion in a mammal and preferably in a human. The use includes the step of application of the coated active ingredient formulation according to the invention or the liquid medical composition according to the invention to the mammal and preferably to the human. In this case, the application can take place before, during or after the physical exertion. It preferably takes place before the physical exertion. The use claimed here constitutes a non-therapeutic performance enhancement without promotion of recovery, since it is to be considered proceeding from a non-pathological normal state. In contrast thereto, the aim of a treatment is always the return from a pathological state to the normal state, or the prevention of a pathological state.

In an alternative aspect, the invention relates, here, to the coated active ingredient formulation according to the invention or the liquid medical composition according to the invention in its use for increasing performance in physical exertion or reducing the recovery time after physical exertion in a mammal and preferably in a human. The use includes the step of application of the coated active ingredient formulation according to the invention or the liquid medical composition according to the invention to the mammal and preferably to the human. In this case, the application can take place before, during or after the physical exertion. It preferably takes place before the physical exertion.

In a further aspect, the invention relates to a method for the production of the coated active ingredient formulation according to the invention in the form of microspherules, comprising the following steps:
(A) providing a CO₂-accepting active ingredient in solid or liquid form, wherein the CO₂-accepting active ingredient is advantageously a CO₂-absorbing material;
(B) providing at least one polymer as a carrier plastics;
(C) supplying the CO₂-accepting active ingredient, the at least one polymer from step (B), to an extruder, and optionally additives such as dispersing aids to the extruder, which is preferably a screw extruder;
(D) dispersing the CO₂-accepting active ingredient in the polymer by melting the polymer in the extruder and intensively mixing the CO₂-accepting active ingredient into the molten polymer stream;
(E) extruding the molten mixture from (D) through one or more shaping openings such as a nozzle or a plurality of nozzles;
(F) cooling the extruded plastics strand;
(G) granulating the extruded plastics strand; particularly preferably directly after the shaping opening in a liquid bath;
(H) optionally grinding the plastics granulate from step (G), preferably by means of cryogenic grinding;
(I) surface treatment for pH-neutralisation or removing the exposed CO₂-accepting active ingredient on the particle surface.

In a further aspect, the invention relates to an alternative method for the production of the coated active ingredient formulation according to the invention in the form of microspherules, comprising the following steps:
(A) providing a CO₂-accepting active ingredient in solid or liquid form, wherein the CO₂-accepting active ingredient is advantageously a CO₂-absorbing material;
(B) providing at least one polymer as a carrier plastics;
(C) supplying the CO₂-accepting active ingredient, the at least one polymer from step (B), to an extruder, and optionally additives such as dispersing aids to the extruder, which is preferably a screw extruder;
(D) dispersing the CO₂-accepting active ingredient in the polymer by melting the polymer in the extruder and intensively mixing the CO₂-accepting active ingredient into the molten polymer stream;
(E1) extruding the molten mixture from (D) through one or more shaping openings such as a nozzle or a plurality of nozzles into an aqueous medium for direct cooling and underwater microgranulation of the extrudate strand;
(I) optionally surface treatment for pH-neutralisation or removing the exposed CO₂-accepting active ingredient on the particle surface.

In step (A) of the two above-mentioned methods, the CO₂-accepting active ingredient is provided in solid or liquid form. The core region of the active ingredient formulation is intended to be formed from this. Preferably, the CO₂-accepting active ingredient is a CO₂-absorbing material, such as calcium hydroxide.

In step (B) of the two above-mentioned methods, at least one polymer is provided as the carrier plastics. Said polymer is suitably provided as a solid polymer, which is preferably provided as a granulate. The polymer is suitably a thermoplastic polymer, which is melted in an extruder and thus allows for the active ingredient to be embedded into the polymer carrier matrix.

In step (C), the components provided according to step (A) and step (B), i.e. the CO₂- accepting active ingredient and the at least one polymer, are supplied to an extruder. Optionally, in addition one or more additives can be added here. An example for a possible additive is a dispersing aid. In an extruder, plastically deformable to viscous masses are continuously pressed out, under pressure, from a shaping opening (also referred to as a nozzle).

The extruder is preferably a screw extruder, which can be designed as a single-screw extruder or twin-screw extruder. In this design, the pressure is generated by means of an extruder screw, also referred to for short as a “screw”. It is inserted in what is known as the screw cylinder, the nominal diameter of which is virtually identical to the outside diameter of the screw. The nozzle, as the shaping outlet opening, is located at the front end of the screw cylinder. The drive, which ensures the rotation of the screw, is located at the rear end of the cylinder. The material is mixed thoroughly by the extruder screw, and the thermoplastic polymer is melted by the pressure and optionally by additional supply of heat by means of a heated screw and/or a heated extruder housing, such that a homogeneous distribution of the CO₂-accepting active ingredient particles in the polymer carrier matrix occurs. The screw additionally conveys the material further, in the direction of the nozzle.

In a further advantageous embodiment, the extruder is a planetary roller extruder. This has the advantage that it allows particularly homogeneous mixing of the supplied components.

Due to the processing of the components in the extruder, in step (D) the CO₂-accepting active ingredient is dispersed in the polymer by melting the polymer in the extruder and intensively mixing the CO₂-accepting active ingredient into the molten polymer stream. This takes place above all in what is known as the compression zone of the extruder, in which the material is further compressed by the reduced thread depth of the screw, and thus the pressure required for the discharge in the die is built up.

The components can expediently undergo degassing in the extruder.

Finally, the discharge zone or metering zone ensures a homogeneous material stream towards the die, such that in step (E) the molten mixture from (D) is extruded through one or more shaping openings such as a nozzle or a plurality of nozzles.

Alternatively, in the last-mentioned method, in step (E1) the molten mixture from (D) undergoes dissipating underwater granulation (also referred to as underwater microgranulation) through one or more shaping openings such as a nozzle or a plurality of nozzles, and thus a microgranulate having homogeneous microdistribution is produced in one step. This method has the further advantage that initially homogeneous particles with complete inclusion of the CO₂-accepting active ingredient result. Basic absorber material (such as calcium hydroxide or magnesium hydroxide) unintentionally present on the surface is immediately washed out by spraying the particles into the aqueous liquid.

The extruded and chopped particles can, with the aid of the rheology of the melt and promoted by the prevailing temperature in the water and the contact-less floating in the liquid, be designed substantially more favourably after the chopping. The method has numerous advantages:
1. Reduction of the surface roughness (smoother surface).
2. Fewer absorber particles located on the surface (i.e. they are still surrounded by the polymer matrix, after chopping).
3. Improved particle shape (i.e. usually spherical particles, which results in improved pourability and more uniform uptake kinetics).
4. Smaller particle sizes can be achieved here, compared with other production methods.

Methods and devices for underwater microgranulation are well known to a person skilled in the art, and said person will select and use then according to the specific requirements (e.g. based on plastics type and particle size).

The extrudate, arising as a plastics strand, is cooled in step (F), such that the thermoplastic polymer correspondingly hardens and thereby firmly surrounds the CO₂-accepting active ingredient.

In step (G), the extruded plastics strand is granulated. This preferably takes place directly after the shaping opening, and in this case particularly preferably in a liquid bath used for cooling. The plastics strand can be divided into individual portions here, using a severing or cutting device.

Step (H) is an optional step, and provides for grinding of the plastics granulate from step (G) that is generated in step (G). This grinding preferably takes place by means of cryogenic grinding.

In step (I), surface treatment of the plastics granulate or of the microgranulate takes place, which serves for pH-neutralisation or removal of the exposed CO₂-accepting active ingredient on the particle surface.

By way of this step, the CO₂-accepting material, which, on account of incomplete coating is provided free on the surface of the particles and can thus interact directly with the surrounding environment, undergoes surface treatment. Advantageously, said surface treatment takes place by means of one or more of the following steps:
(i) washing the microspherules in acid, and subsequent pH-neutralisation;
(ii) temporary melting of the microspherules for sealing the CO₂-accepting active ingredient that is exposed at the surface; or
(iii) additionally coating the microspherules.

In the case of a basic CO₂ absorber, such as calcium hydroxide, according to step (i) the basic absorber exposed on the surface can be neutralised by washing the microspherules with acid. By means of the acid-base reaction, the absorber is converted into a (preferably soluble) salt, which can then be removed by optionally following washing steps.

According to step (ii), the microspherules can undergo temporary melting. This is possible in particular in the case of thermoplastic polymers. As the inventors have discovered, this temporary melting brings about sealing of the CO₂ absorber that is exposed on the surface. This thermal step has the advantage that it does not require chemical treatment, and the total amount of CO₂ absorbers that are contained is maintained. In order to prevent a particle agglomeration, the temporary melting of the microspherules takes place in liquid suspension and/or the microspherules are vigorously mixed.

According to step (iii), the microspherules can be provided with an additional coating. As a result, the CO₂ absorbers exposed on the surface can be coated and thus sealed. By means of such a coating, the microspherules can furthermore be purposely modified with respect to further properties such as hydrophobicity, hydrophilicity or biocompatibility.

In an alternative embodiment, the polymer forming the carrier matrix can be used for coating, in order to thereby achieve a homogeneous structure of the microspherule.

It is also conceivable for an excipient and/or additive to furthermore be added to the above-mentioned components in the method according to the invention. For example, surface-active substances, fillers, further flame inhibitors, nucleation agents, oxidation stabilisers, sliding and demoulding aids, dyes and pigments, optionally stabilisers, e.g. against hydrolysis, light, heat or discolouration, inorganic and/or organic fillers, reinforcing agents, and plasticisers are mentioned. Suitable excipients and additives can be found for example in the Kunststoffhandbuch [Plastics compendium], Volume VIII “Polyester”, Gerhard W. Becker and Dietrich Braun, Carl Hanser Verlag, Munich, Vienna, 1993.

It can furthermore be provided for a bicomponent fibre to be produced in the extrusion method. A bicomponent fibre of this kind consists of two polymers which are firmly but separably connected to one another and have different chemical and/or physical structures. There is a plurality of geometric arrangements of the polymers, wherein, however, the following four types of fibre cross-section are particularly advantageous: a) side-by-side (SS type); b) shell/core (MK type); c) matrix/fibrils (MF type); d) cake slices (also referred to as orange slice-type).

It can advantageously be provided, in the method according to the invention, that the microspherules are dispersed in a carrier solution in a further step.

In a second aspect, the invention relates to microspherules which can be or are produced by means of the extrusion method according to the invention that is described above.

Microspherules can be produced by the extrusion method which have an extremely homogeneous particle size distribution. This ensures a purposeful controllability of the CO₂-uptake by the active ingredient.

Furthermore, the extrusion method allows for large-scale production of the microspherules. The intensive mixing in the extruder results in a homogeneous distribution of the CO₂ absorber in the polymer matrix, which is associated with an advantageous CO₂-uptake kinetics in the in vivo application.

Due to embedding in a polymer matrix, the resulting microspherules are readily distributable and/or storage-stable. Their properties sort in the end formulations can thus be achieved without problem.

In a further possibility, it can be provided that the microspherules have a round, ellipsoidal, irregular, or elongate shape.

An elongate shape can be defined by the aspect ratio, wherein this is defined as the ratio of the length of the microspherule to the greatest lateral extension. In this case, an elongate shape has an aspect ratio of 4:1, preferably of 3:1, and particularly preferably of 2.5:1.

According to a further advantage, it can be provided that the microspherules according to the invention have an outer surface that consists entirely of polymer material.

It can advantageously be provided, in the case of the invention, that the microspherules, in the case of a spherical shape, have a particle size of between 50 and 1000 μm and preferably of between 100 and 500 μm. Particles of a size of smaller than 500 μm have the advantage that they pass quickly through the stomach. Liquids leave the stomach relatively quickly (half-life of water on empty stomach: 10-20 minutes), and this also applies for particles of a small particle size of <500 μm. The particle size of the microspherules can for example be 75, 100, 125, 150, 175, 200, 225, 250, 275, 300, 325, 350, 375, 400, 425, 450 or 475 μm.

In the case of elongate particles, which preferably have a circular, trilobal or square cross-section, it is preferred for the particles to have a diameter of 50 to 800 μm and a length of 4 mm.

### Definitions

In the present document, the term “polymer” includes a collective of macromolecules which are chemically homogeneous but differ with respect to degree of polymerisation, molar mass and chain length, which was produced by a polyreaction (polymerisation, polyaddition, polycondensation). Furthermore, the term also includes derivatives of such a collective of macromolecules from polyreactions, i.e. compounds which were obtained by reactions, for example additions or substitutions, of functional groups on specified macromolecules, and which can be chemically homogeneous or chemically inhomogeneous. The term furthermore includes copolymers and what are known as prepolymers, i.e. reactive oligomer pre-adducts, the functional groups of which are involved in the construction of macromolecules.

In the present document, the term “copolymer” designates a polymer which is composed of two or more different types of monomer units. Thus, the copolymer differs from a homopolymer, which is constructed of just one (real or imaginary) monomer type, and correspondingly has just one repeat unit. Copolymers can be divided into five classes, specifically
1.) statistical copolymers, in which the distribution of the two monomers in the chain follows a statistical distribution,
2.) gradient copolymers, which are in principle similar to the statistical copolymers, in which, however, the fraction of one monomer increases and that of the other decreases in the course of the chain,
3.) alternating copolymers, in which the two monomers alternate,
4.) block copolymers and segment copolymers, which consist of longer sequences or blocks of each monomer, and
5.) graft copolymers, in which blocks of one monomer are grafted to the scaffold (backbone) of another monomer.

In the present document, “molecular weight” is understood to mean the molar mass (in grams per mole or in Daltons) of a molecule. In the present document, “average molecular weight” is always understood to mean the number average of the molecular weight distribution Mₙ (number average). In this case, the term “number-average molar mass” is also used as a synonym for “average molecular weight”.

In the present document, the term “solvents” means compounds as are listed as organic solvents in C D Römpp Chemie Lexikon [Chemistry Lexicon], 9th edition, Version 1.0, Georg Thieme Verlag, Stuttgart 1995. The polyols used according to the invention are not covered by this definition, although the function as solvents for the monomers and also the low-molecular weight polymers resulting from radical polymerisation.

In the present document, “solid” is understood to mean substances which do not change their shape without external action, or can be deformed only with difficulty; in particular, they are, however, not flowable. Correspondingly, “liquid” is understood to mean substances which can be deformed and are flowable, which also includes highly viscous and pasty substances.

It is explicitly noted that, in the context of the patent application present here, indefinite articles and indefinite numbers such as “one . . . ”, “two . . . ”, etc., should generally be understood as minimum specifications, i.e. as “at least one . . . ”, “at least two . . . ”, etc., unless a particular point emerges for example from the context or the specific text to the effect that for example there only “exactly one . . . ”, “exactly two . . . ”, etc. should be meant. Furthermore, all numbers and specifications regarding method parameters and/or device parameters are to be understood in the technical sense, i.e. are to be understood as being provided with the conventional tolerances. It also may not be concluded from the explicit specification of the restriction “at least” or the like that, in the case of the simple use of “one”, i.e. without the specification of “at least” or the like, “exactly one” is meant.

Unless otherwise specified, in the present document the % specifications are weight percent specifications.

The embodiments shown here are merely examples for the present invention, and therefore should not be understood as limiting. Alternative embodiments taken into account by a person skilled in the art are likewise covered by the scope of protection of the present invention.

### Embodiments

### 1. Production of the Microspherules According to the Invention

8 g/min LD-PE having a melting temperature of 242 degrees Celsius is introduced into a sidestream extruder, in the main stream. The main spindle speed is 175 rpm. The extruder comprises six heating zones in which the temperatures of 220, 230, 240, 250, 250 and 250 degrees Celsius, respectively, are set. The sidestream extruder adds 7.4 g/min powdered Ca(OH)₂ at a speed of 170 rpm. The strand is spun out in demineralised water through a 6 mm nozzle having a circular cross-section. In the subsequent granulator, the strand is cut into spherules of 2 mm in length. A further comminution takes place in a cryo-grinder of the Fritsch Pulverisette 14 type at a speed of 20,000 rpm and grinding discs 2.0, with continuous supply of liquid nitrogen, such that microspherules are obtained. The fraction having the desired grain size of 100 to 600 μm is isolated by sieving.

The microspherules of grain size 100 to 600 μm are suspended in a 0.2 wt. % agar-agar solution in a concentration of 25 wt. %. By adding 30% hydrochloric acid, open Ca(OH)₂ particles are neutralised and the pH of the suspension is set to 7.40.

### 2. In Vitro CO₂-Uptake by the Formulation According to the Invention

In order to analyse the CO₂-uptake of the produced suspension, 1 ml thereof is added to a container of 700 ml volume, which contains 9000 ppm CO₂ in N₂. Measuring the CO₂ concentration in the gas mixture every 60 seconds makes it possible to determine the absorption capacity and the absorption kinetics.

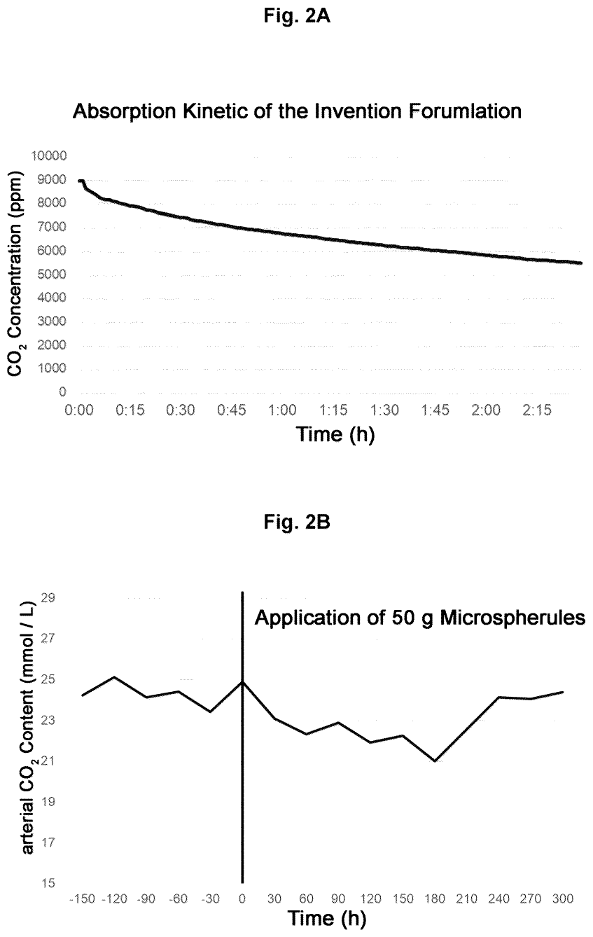

The results are shown in FIG. 2A. Saturation of the microspherules according to the invention is identified after a time period of 90 minutes, such that a retard effect with respect to the CO₂-uptake is achieved by the permeability properties of LDPE.

For the present embodiment, a CO₂ acceptance capacity of 3±0.82 mg CO₂/g microspherules was determined.

### 3. In Vivo CO₂-Uptake by the Formulation According to the Invention

For testing the formulation according to the invention in vivo, the Aachen Mini-Pig was used as an animal model. The animals are ventilated in deep anaesthesia with constant CO₂ elimination via the lungs. Following application of 50 g of the microspherules, which were produced according to Example 1, the CO₂ content of the arterial blood was determined at intervals of 15 minutes, using a duodenal tube.

The content of arterial CO₂ as a function of time is shown in FIG. 2B. Following application, there was a reduction in the arterial CO₂ content from 25 mmol/l to 21 mmol/l for a time period of 3 hours, and a subsequent slow increase in the CO₂ content in the arterial blood, up to the starting value.

These animal tests made it possible to confirm the effectiveness of the formulation according to the invention in mammals.

### 4. Production of the Galenic Sample A

48 wt. % calcium hydroxide was mixed with 52 wt. % low-density polyethylene (LD-PE), extruded into a water bath, and granulated to cylindrical pieces of 1 cm³. Subsequently, the pieces were cryo-comminuted to a diameter of approximately 0.5 mm. The particles thus obtained were stirred in ultrapure water and the pH brought to 7.40 by adding hydrochloric acid. Following sieving, 150 g of the particles were suspended in 1 litre of a solution of 0.2 wt. % agar in ultrapure water, and the pH was set to 7.40 by adding hydrochloric acid.

The CO₂ absorption of the suspension was determined by mixing the suspension in carbonic acid-containing water and stirring for 24 hours in a closed container. 1 g of particles of the galenic sample A absorbed 0.03 g CO₂.

### 5. Production of the Galenic Sample B

30 wt. % calcium hydroxide was incorporated into 70 wt. % high-density polyethylene (HD-PE) and further processed by means of underwater microgranulation, such that particles having a diameter of approximately 0.5 mm resulted. The particles were stirred for 24 hours in a solution of commercially available descaling agent, and rinsed with ultrapure water. Following sieving, 150 g of the particles were suspended in 1 litre of a solution of 0.2 wt. % agar in ultrapure water, and the pH was set to 7.40 by adding hydrochloric acid.

The CO₂ absorption of the suspension was assessed by mixing the suspension in carbonic acid-containing water and stirring for 24 hours in a closed container. 1 g of the particles of the galenic sample B absorbed 0.02 g CO₂.

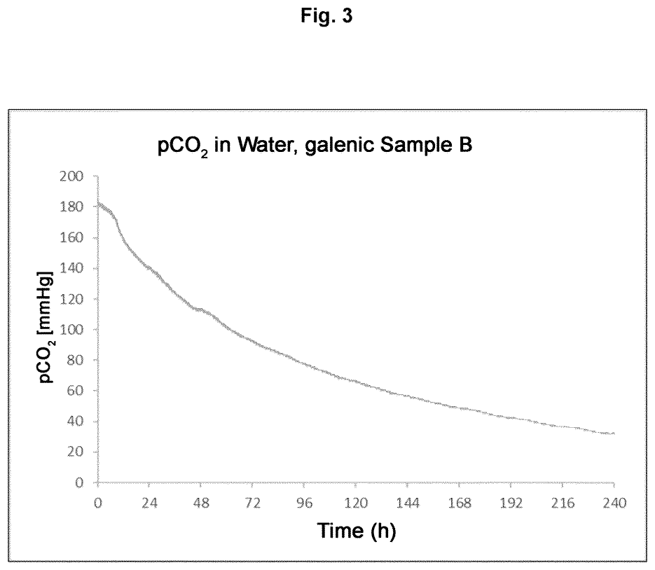

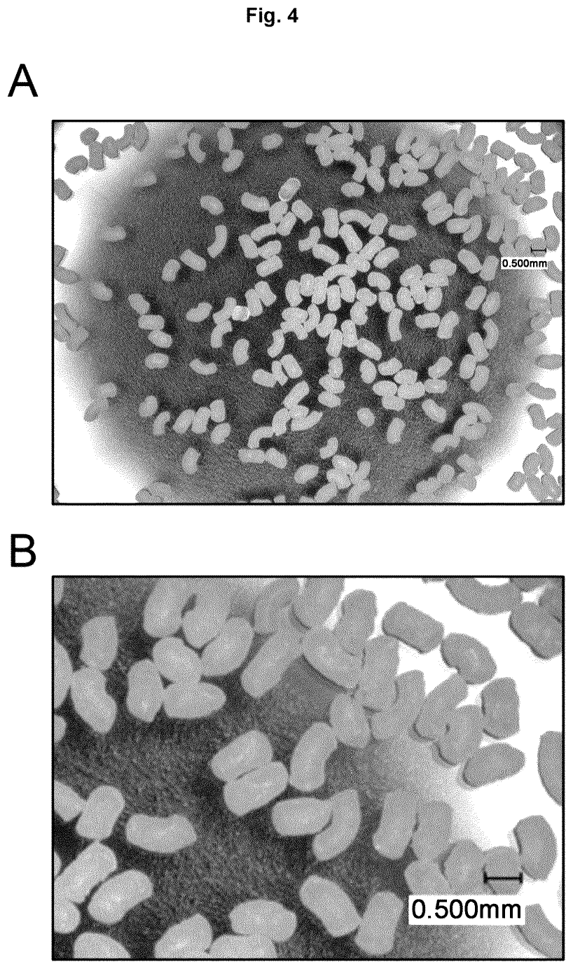

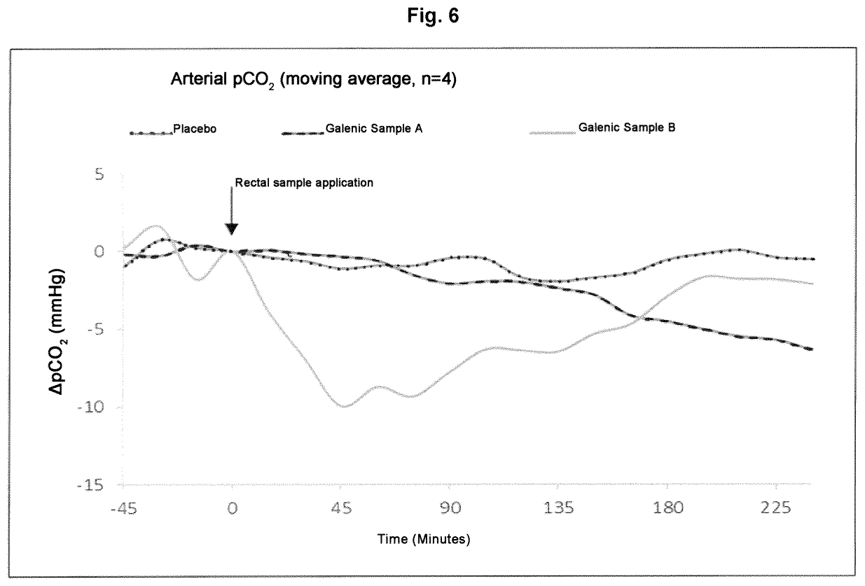

In view of the results from the in vivo experiments (see point 8 and FIG. 6), the kinetics of the CO₂ absorption of galenic Example 2 was tested in carbonic acid-containing water. The results are shown in FIG. 3. Due to the CO₂ absorption of the sample B, a continuous reduction of the CO₂ concentration in the solution is observed, which continues until the last measured timepoint of 240 min and reaches approx. 30 mmHg there. Furthermore, the homogeneity of the particles was demonstrated by means of a micrograph (see FIG. 4).

### 6. Production of the Galenic Sample C

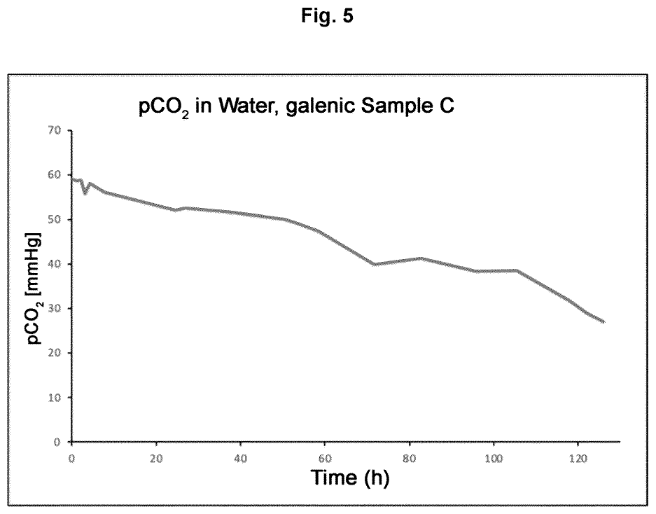

30 wt. % magnesium hydroxide was incorporated into 70 wt. % low-density polyethylene (LD-PE), extruded into a water bath, and continuously cast to cylindrical pieces of 1.4 cm3. The particles were stirred for 48 hours in a solution of commercially available descaling agent, and rinsed with ultrapure water. After the sieving, the CO₂ absorption of the particles was assessed by mixing the suspension in carbonic acid-containing water and stirring in a closed container. 1 g of the particles of the galenic sample C absorbed 0.02 g CO₂. The kinetics of the CO₂ absorption of the galenic sample C was tested in carbonic acid-containing water. The results are shown in FIG. 5. Due to the CO₂ absorption of the sample B, a continuous reduction of the CO₂ concentration in the solution is observed, which already represents a reduction of the CO₂ concentration by virtually 50% after 120 minutes (60 mmHg to 30 mmHg).

7. Production of a Comparative Sample (“Placebo”)

High-density polyethylene was extruded by means of underwater microgranulation, wherein particles having a diameter of approximately 0.5 mm resulted. The particles were stirred for 24 hours in a solution of commercially available descaling agent, and rinsed with ultrapure water. Following sieving, 150 g of the particles were suspended in 1 litre of a solution of 0.2 wt. % agar in ultrapure water, and the pH was set to 7.40 by adding hydrochloric acid. The placebo sample did not exhibit any CO₂ absorption from carbonated water.

8. In Vivo Testing of the Samples

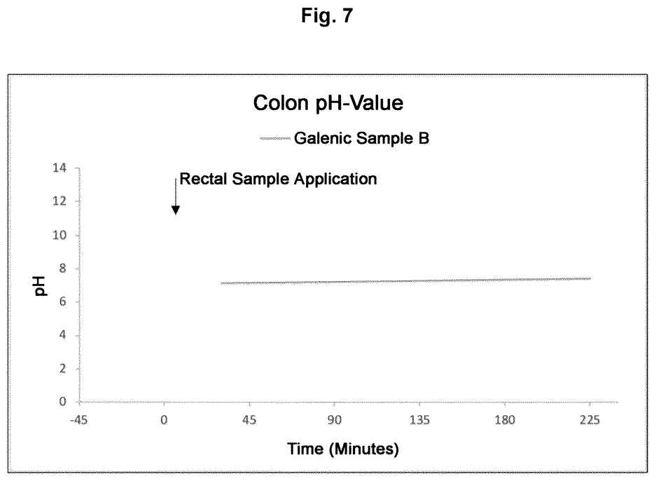

The galenic samples A and B and the placebo sample were tested on pigs. The pigs were put under general anaesthetic; after the arterial pCO₂ value had been set to approximately 50 mmHg. the settings of the ventilator device were kept constant throughout the entire test. The above-mentioned suspension of 150 mg of each galenic version in 1 l of ultrapure water thickened with agar was administered rectally. The arterial pCO₂ values were measured every 15 minutes throughout the entire test duration. The results are shown in FIG. 6. Both galenic samples led to a reduction in the arterial pCO₂ values, while the placebo did not influence the pCO₂ values. The kinetics of the galenic sample B is to be assessed as particularly advantageous, since it leads, in vivo, to a quick reductio of the CO₂ concentration (reduced by 10 mmHg within 45 minutes). For the galenic sample B, the pH of the large intestine was measured regularly during the experiment. The results are shown in FIG. 7. In the case of a leakage of the particles, the pH in the colon should increase significantly. As the measurement results show, the pH remained constant during the entire course of the test, which indicates a reliable and constant inclusion of the basic CO₂ absorber. In this case, the pH is in the physiological range of 7.13 to 7.42.

Different embodiments of the invention will be described in the following with reference to the drawings, in which:

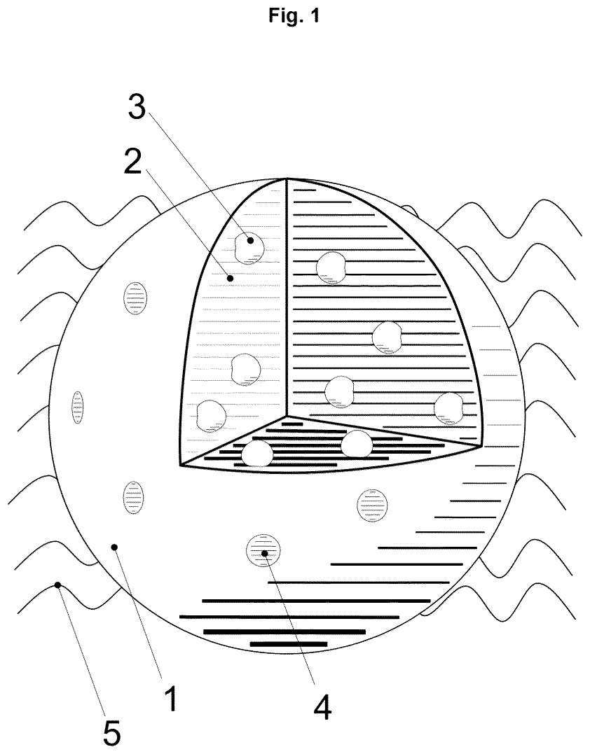

FIG. 1 is a schematic view of a microspherule according to the invention.

FIG. 2A shows results for in vitro uptake of CO₂ by the microspherules according to the invention.

FIG. 2B shows results for in vivo uptake of CO₂ by the microspherules according to the invention.

FIG. 3: shows the kinetics of the in vitro CO₂ absorption for galenic sample B.

FIG. 4: A: is a photomicrograph image of the galenic sample B, showing an enlargement of a detail in B.

FIG. 5: shows the kinetics of the in vitro CO₂ absorption for galenic sample C.

FIG. 6: shows the in vivo testing of the galenic samples A and B compared with the placebo, by measuring the arterial pCO₂ value after rectal application of the samples in pigs.

FIG. 7: shows the in vivo testing for tolerability of the galenic sample B by measuring the colon pH after rectal application of sample B in pigs.

FIG. 1 is a schematic view of a microspherule 1 according to the invention. In this embodiment, the microspherule 1 assumes a spherical shape, and thus has a particularly advantageous ratio of volume to surface area. It consists of a polymer matrix 2, in which individual particles of CO₂ absorber 3 are included in homogeneously distributed form. The CO₂ absorbers located on the surface were removed by acid treatment with subsequent washing steps, such that only cavities 4 remained, and thus there is no longer any CO₂ absorber located on the surface. The microspherule 1 is present as a particle in a liquid dispersion medium 5.

### LIST OF REFERENCE SIGNS

1 microspherule
2 polymer matrix
3 CO₂ absorber
4 surface cavity resulting from washed-out CO₂ absorber
5 dispersion medium

1-26. (canceled)

27. A coated active ingredient formulation for the controlled in vivo uptake of CO₂, comprising:
(i) a coating that consists of a wall material, wherein the coating is permeable for CO₂ and optionally also for water vapour; and
(ii) a core region which comprises at least one CO₂-accepting active ingredient.

28. The coated active ingredient formulation according to claim 27, wherein the coating is impermeable to water.

29. The coated active ingredient formulation according to claim 27, wherein the coated active ingredient formulation is selected from the group consisting of:
a liquid core comprising the CO₂-accepting active ingredient having a membrane envelope for forming a liposome or niosome;
a solid core comprising a CO₂-accepting active ingredient having a single-layer coating;
a solid core comprising a CO₂-accepting active ingredient having a multi-layer coating; and
a microcapsule.

30. The coated active ingredient formulation according to claim 29, wherein the coated active ingredient formuation is the microcapsule, wherein the microcapsule is a microspherule, and wherein the CO₂-accepting active ingredient is incorporated in solid dispersed form or in dissolved form in a polymer carrier matrix.

31. The coated active ingredient formulation according to claim 27, wherein the CO₂-accepting active ingredient is selected from the group consisting of a CO₂-absorbing material, a CO₂-adsorbing material, a CO₂-dissolving material, and a CO₂-converting enzyme.

32. The coated active ingredient formulation according to claim 27, wherein the coating has a CO₂ permeability of 0.004 to 2,500×10⁻¹³ cm³ cm cm⁻² Pa s⁻¹.

33. The coated active ingredient formulation according to claim 27, wherein the coating has a water vapour permeability of 0.009 to 32,000×10⁻¹³ cm³ cm cm⁻² Pa s⁻¹.

34. The coated active ingredient formulation according to claim 27, wherein the coating has a mass fraction of 10 to 95 wt. % based on the weight of the coated active ingredient formulation.

35. The coated active ingredient formulation according to claim 27, wherein the coating has an average layer thickness of 10 to 500 μm.

36. The coated active ingredient formulation according to claim 27, wherein the wall material is selected from the group consisting of:
poly(organo)siloxane;
polyolefin;
polyester;
polybutylene succinate;
polybutylene adipate terephthalate (PBAT);
polyethylene;
polylactide (PLA);
poly(glycolic acid) (PGA);
poly-lactic-co-glycolic-acid (PLGA); and
lipids.

37. The coated active ingredient formulation according to claim 27, wherein the CO₂-accepting active ingredient is one of:
(a) a CO₂-absorbing material selected from the group consisting of inorganic hydroxide compounds, lithium peroxide, and mixtures thereof; or
(b) a CO₂-adsorbing material selected from the group consisting of active carbon, a molecular sieve, silica, metalorganic frameworks (MOFs); or
(c) a CO₂-dissolving material selected from the group consisting of a highly-eutectic solvent and an ionic fluid; or
(d) a CO₂-converting enzyme.

38. The coated active ingredient formulation according to claim 37, wherein the CO₂-absorbing material comprises calcium hydroxide and optionally an effective amount, capable of absorbing carbon dioxide, of a hygroscopic or deliquescent humectant, and wherein the calcium hydroxide is substantially free of sodium and potassium hydroxide.

39. The coated active ingredient formulation according to claim 27, wherein the CO₂-absorbing material has a CO₂ acceptance capacity of more than 10 mg CO₂/g active ingredient formulation.

40. A liquid medical composition as a dispersion comprising a coated active ingredient formulation according to claim 27, and to a pharmaceutically safe aqueous carrier fluid.

41. The liquid medical composition according to claim 40, wherein the liquid medical composition is provided for intraperitoneal or enteral application via a tube.

42. A method for prophylaxis or treatment of a disease of the respiratory system of a patient, the method comprising administering a coated active ingredient formulation according to claim 27 to the patient.

43. The method according to claim 42, wherein the disease of the respiratory system is one of:
(a) a chronic obstructive pulmonary disease;
(b) asthma;
(c) cystic fibrosis;
(d) acute respiratory distress syndrome;
(e) hypercapnia;
(f) pneumonia;
(g) lung cancer;
(h) pulmonary fibrosis;
(i) a respiratory disorder according to medical provisions pursuant to ICD-10 J95;
(j) respiratory failure pursuant to ICD-10 J96;
(i) a respiratory disorder pursuant to ICD-10 J97;
(l) a respiratory disorder in diseases classified elsewhere pursuant to ICD-10, J99; or
(m) immature lungs.

44. A method for the induction of hypocapnia in a patient, the method comprising administering a coated active ingredient formulation according to claim 27 the patient.

45. A method for increasing performance in physical exertion or reducing recovery time after physical exertion in a mammal, the method comprising administering a coated active ingredient formulation according to claim 27 to the mammal.

46. The method for producing microspherules according to 30. the method comprising the following steps:
(A) providing a CO₂-accepting active ingredient in solid or liquid form;
(B) providing at least one polymer as a carrier plastics;
(C) supplying the CO₂-accepting active ingredient from step (A), the at least one polymer from step (B), and optionally additives to an extruder;
(D) dispersing the CO₂-accepting active ingredient in the polymer by melting the polymer in the extruder and intensively mixing the CO₂-accepting active ingredient into a molten mixture;
(E) extruding the molten mixture from step (D) through one or more shaping openings to form one or more extruded plastics strands;
(F) actively or passively cooling the one or more extruded plastics strands;
(G) granulating the one or more extruded plastics strands to obtain a plastics granulate, and forming microspherules therefrom;
(H) optionally grinding the plastics granulate from step (G) prior to formation of the microspherules; and
(I) performing a surface treatment for neutralising or removing exposed CO₂-accepting active ingredient on the surface of the microspherules.

47. The method according to claim 46, wherein the surface treatment of the microspherules for pH-neutralisation of the exposed CO₂-absorber is performed by one or more of the following steps:
(i) washing the microspherules in acid, and subsequent pH-neutralisation;
(ii) temporary melting of the microspherules for sealing the CO₂-accepting active ingredient that is exposed at the surface; and
(iii) additionally coating the microspherules.

48. The method according to claim 40, wherein the microspherules are dispersed in a carrier solution in a further step.

49. Microspherules produced by a method according to claim 40.

50. The microspherules according to claim 49, wherein the microspherules have a round, ellipsoidal, irregular or elongate shape.

51. The microspherules according to claim 49, wherein the microspherules have a surface that consists completely of a polymer material.

52. The microspherules according to claim 49, wherein the microspherules have a particle size of between 50 and 1000 μm.
